(19) United States
(12) Patent Application Publication
(10) Pub. No.: US 2026/0047758 A1
(43) Pub. Date: Feb. 19, 2026

(54) SYSTEMS AND METHODS FOR TREATING AN OPACITY IN AN EYE

(71) Applicant: LUMENIS BE LTD., YOKNEAM (IL)

(72) Inventors: May Kleinman Ben Tsvi; Adrian Graus

(21) Appl. No.: 19/100,818

(22) PCT Filed: Aug. 2, 2023
(86) PCT No.: PCT/IL2023/050804

§ 371 Date: Feb. 3, 2025

Related U.S. Application Data
(60) Provisional application No. 63/394,731, filed on Aug. 3, 2022.

(51) Int. Cl. A61B 3/125; A61B 3/00; A61B 3/10; A61F 9/008

(52) U.S. Cl. CPC A61B 3/125; A61B 3/0008; A61B 3/0033; A61B 3/1005; A61F 9/008; A61F 2009/00874

(57) ABSTRACT

System and method for using in treatment of an opacity within an eye are presented, the system comprising an input utility configured to receive input data comprising image data of the eye being indicative of a convergence spot of first and second aiming beams entering the eye and converging at location of an opacity within the eye, and of first and second spots formed on the retina by respectively the first and second aiming beams; a processing utility configured to process the input data, the processing comprises processing the image data and determining an inter-spot distance between the first and second spots formed on the retina, and utilizing the inter-spot distance and a model of the eye and determining a first distance between the opacity and the retina and a second distance between the opacity and the crystalline lens of the eye; and an output utility configured and operable to generate output data indicative of opacity treatment recommendation based on the first and second distances.

### TECHNOLOGICAL FIELD

The present invention is in the medical field and relates specifically to ophthalmology, and more specifically to systems and methods for treating opacities, the so-called floaters, located within the vitreous humor of the eye.

### BACKGROUND

Eye floaters are opacities in the vision field that may be caused by different conditions such as inflammations and bleeding within the eye, retina tearing and age-related changes occurring when the vitreous becomes more liquid and microscopic fibers within the vitreous cast shadows on the retina.

Eye floaters are typically treated using a laser system, for example the Digital Duet system by Lumenis Be Ltd., shown in FIG. 1, for irradiating and removing the floaters. The laser treatment is done using the photo disruption (PD) mode known as “YAG” (named after the laser crystal material Nd: YAG which stands for Neodymium-doped Yttrium Aluminum Garnet) and having a 1064 nm treatment beam which is not visible to the human eye.

The treating physician uses the laser beam to break the opacities into small parts that later sink due to the gravitation. The laser beam in YAG mode converges strongly to about 8 μm spot with high fluence to enable this blast.

The exemplified system of FIG. 1 has two aiming beams, shown in FIG. 2, riding on top of the YAG beam, to enable the physician to track the invisible laser beam. The user sees two spots of the aiming beams (red spots) overlapping and converging into one spot at the focal plane which is where the floater is located, thus knowing where to fire the YAG treatment beam. It is a common practice to use energy in the range of 2.5-7.5 mJ, and the energy level can vary depending on the opacity size and thickness.

One of the risks when treating these opacities in the vitreous is to unintentionally irradiate the crystalline lens or the retina with the laser beam. The thumb rule is to leave a safe zone of a few millimeters, e.g. about 3 mm, away from the crystalline lens and the retina when treating opacities. A less-experienced user might not correctly estimate the position of the opacity and its distance from the crystalline lens and/or the retina. This may lead to wrong treatment decisions by treating an opacity which is not in the safe zone or not treating an opacity which is in the safe zone.

### SUMMARY OF THE INVENTION

The present invention provides a technique for objectively and accurately determining whether an opacity (or interchangeably “floater”) is in safe zone and can be safely treated by laser systems, including conventional systems, used for this purpose.

In accordance with a first aspect of the invention, there is provided a system for using in treatment of an opacity within an eye, the system comprising: an input utility configured and operable to receive input data, the input data comprising image data of the eye being indicative of a convergence spot of first and second aiming beams entering the eye and converging at location of an opacity within the eye, and of first and second spots formed on the retina by respectively the first and second aiming beams;
a processing utility configured and operable to process the input data, the processing comprises:
   processing the image data and determining an inter-spot distance between the first and second spots formed on the retina; and
   utilizing the inter-spot distance and a model of the eye and determining a first distance between the opacity and the retina and a second distance between the opacity and the crystalline lens; and
an output utility configured and operable to generate output data indicative of opacity treatment recommendation based on the first and second distances.

The image data can be obtained by using a camera located on the laser system of FIG. 1, for example a camera integrated in the slit lamp of the system.

In some embodiments, the opacity treatment recommendation is to treat the opacity when both of said first and second distances are above a predetermined value, and to refrain from treating the opacity when either of the first or second distances is below the predetermined value. The predetermined value may be, for example, up to 3 millimeters.

In some embodiments, the input data comprises data indicative of a contact lens used in front of the eye when said image data of the eye is acquired, said processing of the input data by the processing utility comprises processing the data indicative of the contact lens for determining the first and second distances. In some embodiments, the input utility may be configured to provide data of a plurality of contact lenses, enabling a user to choose the contact lens in use.

In some embodiments, the input data comprises eye prescription data of an individual undergoing the treatment, said processing of the input data by the processing utility comprises processing the eye prescription data for determining the first and second distances.

In accordance with another aspect of the invention, there is provided a device for treating an opacity within an eye, the device comprising:
a system as described above,
a light system for generating and directing first and second aiming beams such as to converge at location of an opacity within the eye and form respective first and second spots on the retina of the eye,
a laser system for irradiating the opacity with a laser treatment beam in order to destroy and remove the opacity, the laser treatment beam trajectory being defined by trajectories of the first and second aiming beams,
a controller for activating the light system and the laser system, and
a camera for acquiring image data of the eye, the image data comprising data indicative of trajectories of the first and second aiming beams within the eye.

In some embodiments, the controller is configured and operable to receive the output data indicative of opacity treatment recommendation, and controllably activate said laser source based on the opacity treatment recommendation.

In accordance with another aspect of the invention, there is provided a method for using in treatment of an opacity within an eye, the method comprising:
receiving input data, the input data comprising image data of the eye being indicative of a convergence spot of first and second aiming beams entering the eye and converging at location of an opacity within the eye, and of first and second spots formed on the retina by respectively the first and second aiming beams;
processing the input data, the processing comprises:
   processing the image data and determining an inter-spot distance between the first and second spots formed on the retina; and
   utilizing the inter-spot distance and a model of the eye and determining a first distance between the opacity and the retina and a second distance between the opacity and the crystalline lens; and
generating output data indicative of opacity treatment recommendation based on the first and second distances.

In some embodiments, the opacity treatment recommendation is to treat the opacity when both of said first and second distances are above a predetermined value, and to refrain from treating the opacity when either of the first or second distances is below the predetermined value. The predetermined value may be, for example, up to 3 millimeters.

In some embodiments, the receiving of the input data comprises receiving data indicative of a contact lens used in front of the eye when said image data of the eye is acquired, said processing of the input data comprises processing the data indicative of the contact lens for determining the first and second distances.

In some embodiments, the method comprises providing data of a plurality of contact lenses, enabling a user to choose the contact lens in use.

In some embodiments, the receiving of the input data comprises receiving eye prescription data of an individual undergoing the treatment, said processing of the input data comprises processing the eye prescription data for determining the first and second distances.

In accordance with another aspect of the invention, there is provided a method for treating an opacity within an eye, the method comprising:
generating and directing first and second aiming beams such as to converge at location of an opacity within the eye and form respective first and second spots on the retina of the eye,
acquiring image data of the eye, the image data comprising data indicative of trajectories of the first and second aiming beams within the eye,
processing the image data and determining an inter-spot distance between first and second spots formed on the retina by respectively the first and second aiming beams,
utilizing the inter-spot distance and a model of the eye and determining a first distance between the opacity and the retina and a second distance between the opacity and the crystalline lens; and
irradiating the opacity with a laser treatment beam in order to destroy and remove the opacity upon determining that both of said first and second distances are above a predetermined value.

In some embodiments, the irradiating of the opacity is done automatically upon determining that both of said first and second distances are above the predetermined value.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 illustrates an example of a system used for treating floaters;

FIG. 2 illustrates trajectories of aiming beams generated and used with the system of FIG. 1;

FIG. 3 exemplifies a non-limiting example of a system for use in treating a floater, according to the invention;

FIG. 4 exemplifies a non-limiting example of a method for use in treating a floater, according to the invention;

FIG. 5 illustrates a non-limiting example of Eye model that can be used with the present invention; and

FIGS. 6-8 illustrate trajectories of aiming beams in the eye and interactions between the aiming beams and optical surfaces and floaters within the eye.

### DETAILED DESCRIPTION

The present invention provides a technique for accurate, objective, determination of the safe zone for treating eye floaters, hence providing a guided and safe treatment. The systems and methods described herein are operable to provide a floater treatment recommendation telling the user whether it is safe or not to treat the floater. In some embodiments, the systems are operable to treat the floater automatically based on the treatment recommendation.

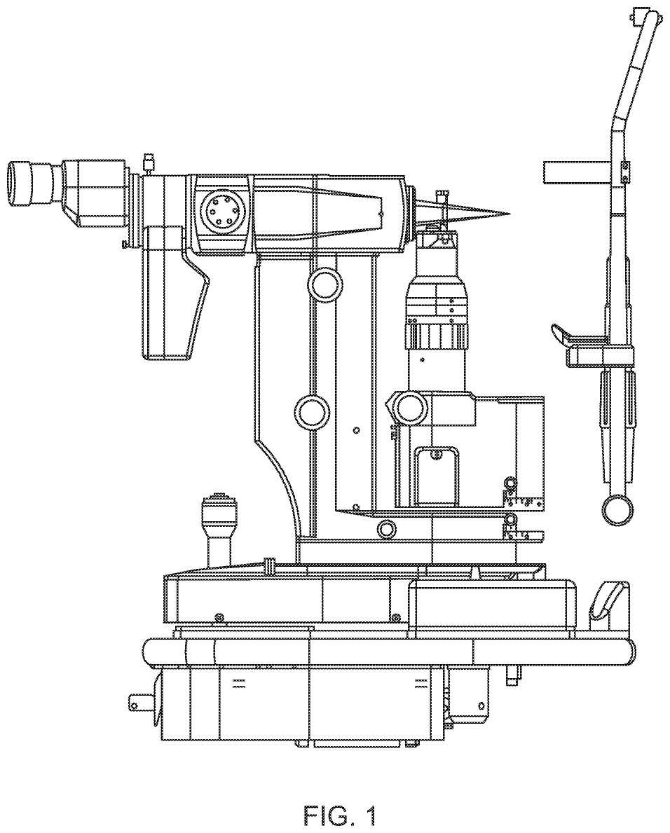

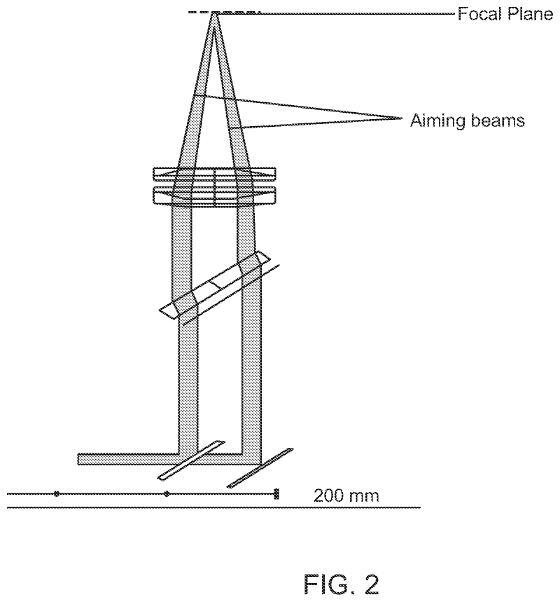

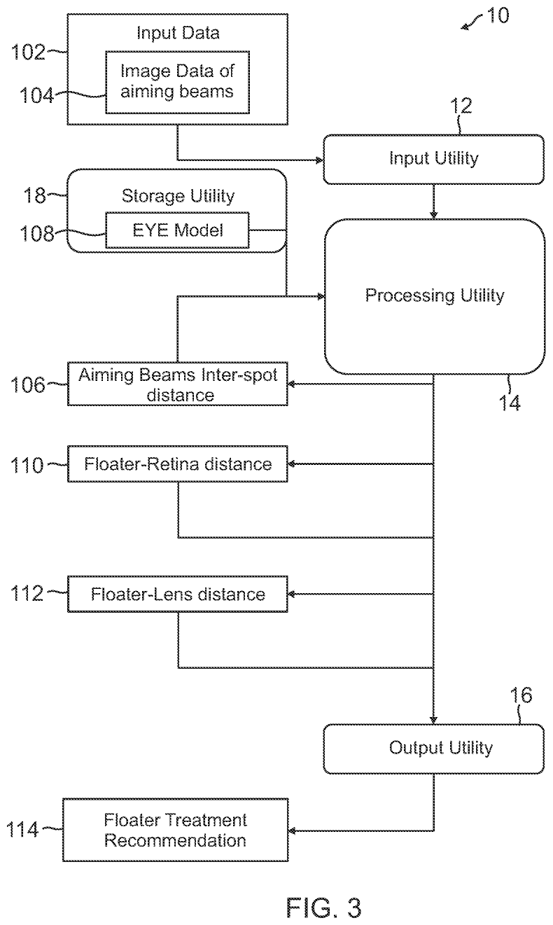

Reference is made to FIG. 3 illustrating a non-limiting exemplary embodiment of a system 10 for using in treatment of floaters, in accordance with the present invention. The system 10 includes an input utility 12, a processing utility 14 and an output utility 16. The system 10 can be implemented as software components running on a computerized system and/or as a firmware running on a dedicated treatment device such as the device of FIG. 1.

The input utility 12 is configured and operable to receive input data 102. The input data 102 includes image data 104 of the eye, taken online by a dedicated camera when the two aiming beams, as described above, are illuminated into the eye. The two aiming beams are directed inside the eye to converge into one spot at the treatment plane where the floater is located, with the beams separating again after the treatment plane and hitting the retina at two different points. Accordingly, the image data 104 is indicative of a convergence spot of the first and second aiming beams converging at location of an opacity within the eye, and of first and second spots formed on the retina by respectively the first and second aiming beams.

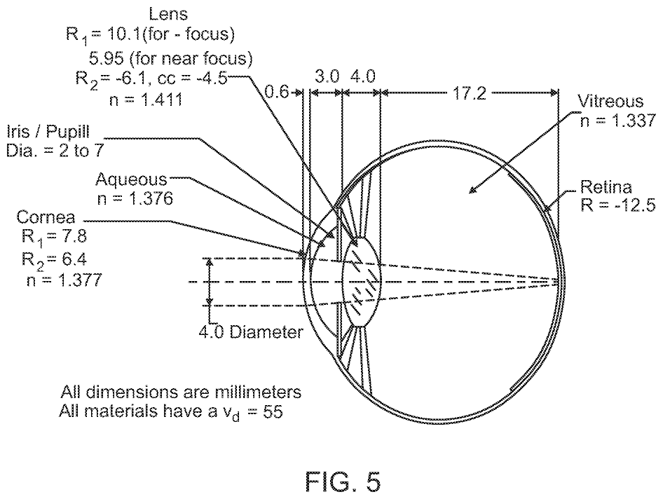

The processing utility 14 is configured and operable to process the input data 102. The processing utility 14 processes the image data 104 and determines an inter-spot distance 106 between the first and second spots formed on the retina by the first and second aiming beams. Then, the processing utility 14 utilizes the inter-spot distance 106 and a model of the eye 108 and determines a first distance 110 between the floater/opacity and the retina and a second distance 112 between the floater/opacity and the crystalline lens. It is noted that the distances 110 and 112 are considered as the distance between the location of the converged spot and the closest point on the retina or the crystalline lens. In some embodiments, the eye model is the walker eye model shown in FIG. 5. The eye model 108, as well as other important data, can be stored in a storage utility 18 and accessible by the processing utility 14. The storage utility 18 can be physical and locally accessible or distant and remotely accessible.

The output utility 16 receives the data of the first and second distances and generates output data indicative of floater/opacity treatment recommendation 114.

In some embodiments, the treatment recommendation 114 is to treat the floater as it is located in a safe zone, or not to treat the floater as it is out of the safe zone. The safe zone is met when both of the first and second distances (110, 112) are above a predetermined value, and is not met when either of the first or second distances is below the predetermined value. In some embodiments, the predetermined value is about 3 millimeters (when “about”as used herein means +/−10%), or up to 3 millimeters.

In some cases, the physician uses a contact lens in front of the eye of the patient when looking at and treating the floater(s). Accordingly, in some embodiments, the input utility is configured to receive optical data indicative of the contact lens used in front of the eye when the image data 104 is acquired, and the processing utility is configured to process the contact lens data in determination of the first and second distances.

As it is somehow a closed list of contact lenses that are used by the physicians, the system, within the storage utility 18, can be configured to store data relating to a plurality of contact lenses, and the input utility 12 can be configured to provide the data of the plurality of contact lenses to the user, e.g. in a drop-down list or similar, and enable the user to choose the contact lens under use.

In some embodiments, the input data 102 also includes prescription data of the patient in case he/she has one. The processing utility processes the prescription data in determination of the first and second distances.

In some embodiments, a device for treating a floater/opacity is provided. The device includes a camera for taking images of the eye when the aiming beams are generated and focused on the floater, a laser system for irradiating the floater whenever it is determined to be in the safe zone, and the system 10 that determines whether the floater is in the safe zone. It is noted that each time the laser usually hits a tiny and pointed part in the vitreous space, e.g. a portion of the floater, and it may be needed to repeat the process by the system 10 to remove large floaters. The user may realign the laser system together with the aiming beams to a new point/portion of the floater, which may have moved meanwhile, take further images and activate the system 10 for calculating whether the new point/portion of the floater is in a safe zone, and finally activate the laser system to fire the new point/portion of the floater when it is in the safe zone, and repeat the process until removing the whole floater. In some embodiments, the device can be programmed to automatically repeat the process described above, i.e. realigning the aiming beams, acquiring images, analyzing the images and determining whether the examined floater or portion of a floater is in the safe zone, and eventually firing the laser beam to remove the floater or the portion of the floater, until the whole floater is removed or is broken into enough small parts that will sink due to gravity.

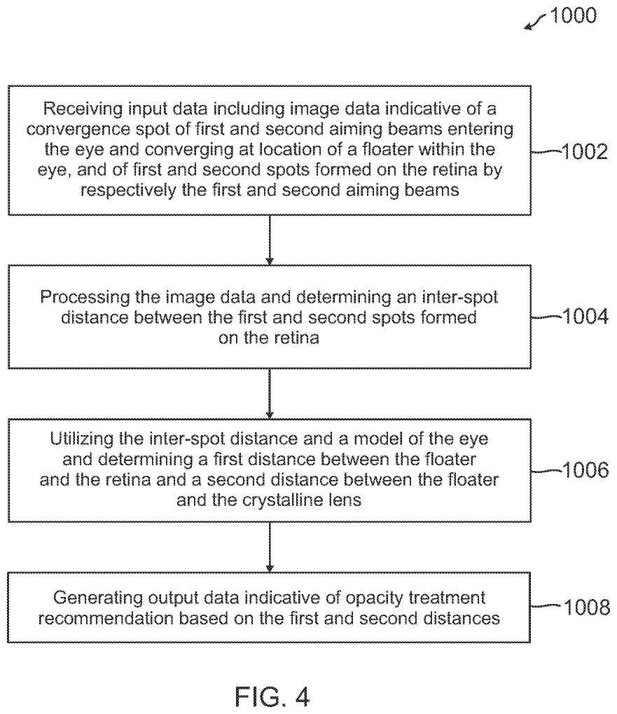

Reference is made to FIG. 4 illustrating a non-limiting exemplary embodiment of a method 1000 for using in treatment of floaters, in accordance with the present invention. At a first step 1002, the method includes receiving input data, the input data include image data of the eye being indicative of a convergence spot of first and second aiming beams entering the eye and converging at location of a floater/opacity within the eye, and of first and second spots formed on the retina by respectively the first and second aiming beams.

At step 1004, the method includes processing the input data. The image data is processed and an inter-spot distance between the first and second spots formed on the retina is determined.

At step 1006, the method includes utilizing the inter-spot distance and a model of the eye and determining a first distance between the floater/opacity and the retina and a second distance between the floater/opacity and the crystalline lens.

At step 1008, the method includes generating output data indicative of opacity treatment recommendation based on the first and second distances. The opacity treatment recommendation can be to treat the opacity when both of said first and second distances are above a predetermined value, or not to treat the opacity when either of the first or second distances is below the predetermined value. In some embodiments, the predetermined value is about 3 millimeters (when “about” as used herein means +/−10%), or up to 3 millimeters.

In some embodiments, the input data includes data indicative of a contact lens used by the physician in front of the eye during the treatment process. The contact lens data is processed for determining the first and second distances.

In some embodiments, the physician is provided with data of a plurality of contact lenses, enabling the physician to choose the contact lens under use.

In some embodiments, when the patient has eye prescription, the eye prescription data is provided and processed for determining the first and second distances.

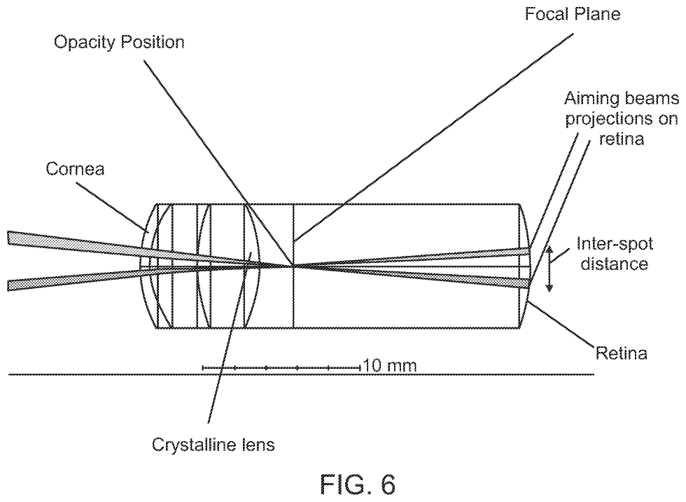

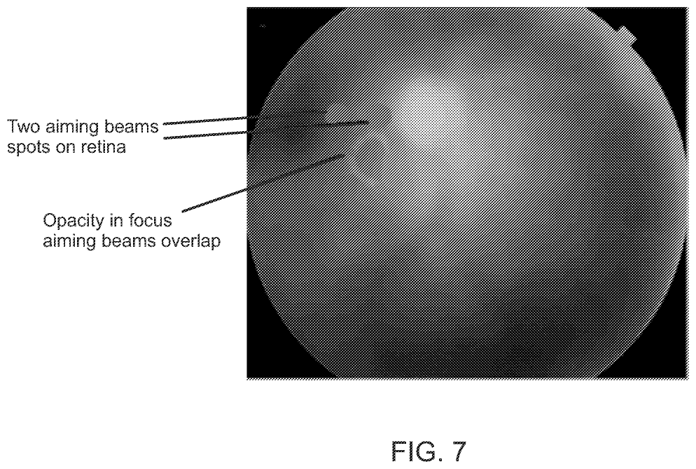

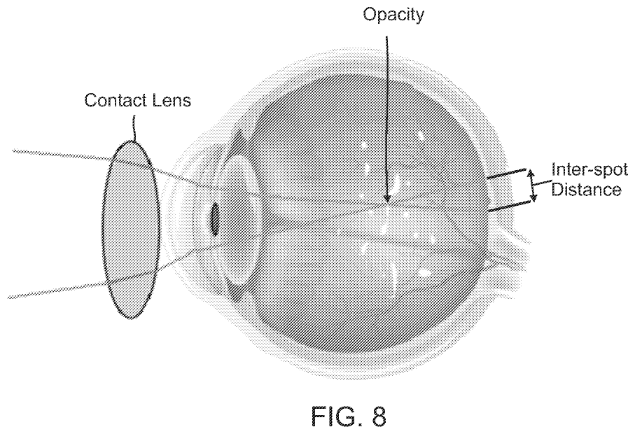

Reference is made to FIG. 6 that exemplifies the optical path of the two aiming beams within the eye. When using the laser system, the physician moves the Slit lamp until seeing the floater at the focal plane/treatment plane. The two aiming beams overlap and converge into one focused spot at the treatment plane, then separate and form two blurred spots on the retina. This can be also seen in FIGS. 7 and 8 illustrating the two aiming beams, the floater and the retina in front and side views respectively.

In the following, description of experiments done by the inventors can be found. The inventors used Zemax (optical design tool) to simulate the optical path of the aiming beams when the slit lamp is aligned so that the opacity is at the focal plane (FIG. 6). In the simulation, Walker model (FIG. 5) that permits to simulate light rays'trajectory within the human eye was used.

When moving the floater/opacity along the vitreous, the inventors can calculate the inter-spot distance between the aiming beams'spots on the retina for each floater/opacity position. A plot of the floater/opacity position vs. the inter-spot distance is obtained and for which a linear regression is done.

The simulation is done using a contact lens used for treating floaters/opacities in the vitreous. There are different optional lenses and the simulation can give a different plot and regression analysis per lens, thus the type of contact lens is taken as a relevant parameter.

The inventors simulated various conditions, such as slit lamp offset or tilt, tilt of the contact lens, and eye sight imperfection like short sight, and found that the impact of these conditions is not significant, except for eye sight imperfection and for which correction plots were calculated, thus the patient prescription is taken as a relevant parameter.

In one non-limiting example, the treatment workflow can be as follows:

The user choses the type of contact lens and feed it into the system, by a suitable GUI.

The user feeds the patient prescription into the system, by a suitable GUI.

The user aligns the slit lamp such that the image is focused on the floater/opacity (the two aiming beams spots overlap in the focal plan, and one can see the blurred spots of the aiming beams reaching the retina in the background).

The user takes an image using the digital camera of the Digital Duet integrated onto the system's slit lamp.

The system calculates the position of the floater/opacity and returns indication if it is in the safe zone

The user fires the floater/opacity if in safe zone or moves to another opacity.

1. A system for use in treatment of an opacity within an eye, the system comprising:
an input utility configured and operable to receive input data, the input data comprising image data of the eye being indicative of a convergence spot of first and second aiming beams entering the eye and converging at location on a focal plane of an opacity within the eye wherein. the first and second aiming beams cross each other to form, first and second spots on the retina by respectively the first and second aiming beams a distance spaced from one another;
a processing utility configured and operable to process the input data, the processing comprises:
processing the image data and determining an inter-spot distance between the first and second spots formed on the retina; and
utilizing the inter-spot distance and a model of the eye and determining a first distance between the opacity and the retina and a second distance between the opacity and the crystalline lens of the eye; and
an output utility configured and operable to generate output data indicative of an opacity treatment recommendation based on the first and second distances.

2. The system according to claim 1, wherein said opacity treatment recommendation is to treat the opacity when both of said first and second distances are above a predetermined value, and to refrain from treating the opacity when either of the first or second distances is below the predetermined value.

3. The system according to claim 2, wherein said predetermined value is up to 3 millimeters.

4. The system according to claim 1, wherein said input data comprises data indicative of a contact lens used in front of the eye when said image data of the eye is acquired, said processing of the input data by the processing utility comprises processing the data indicative of the contact lens for determining the first and second distances.

5. The system according to claim 4, wherein said input utility provides access to data of a plurality of contact lenses, enabling a user to choose the contact lens to use.

6. The system according to claim 1, wherein said input data comprises eye prescription data of an individual undergoing the treatment, said processing of the input data by the processing utility comprises processing the eye prescription data for determining the first and second distances.

7. A device for treating an opacity within an eye, the device comprising:
a light system configured to generate and direct a first and
second aiming beams such as to converge at the focal plane location of an opacity within the eye wherein, the first and second aiming beams cross each other to form respective first and second spots on the retina of the eye at a distance spaced from one another,
a laser system configured to irradiate the opacity with a laser treatment beam to destroy and remove the opacity, the laser treatment beam trajectory being defined by trajectories of the first and second aiming beams,
a camera configured to acquire image data of the eye, the image data comprising data indicative of trajectories of the first and second aiming beams within the eye; and
a controller, when activated, is configured to:
activate the light system;
activate the camera;
receive image data from the camera;
process the image data; and
output data indicative of opacity treatment recommendation based on the processed input data.

8. The device according to claim 7, wherein said controller is configured and operable to receive the output data indicative of an opacity treatment recommendation and controllably activate said laser source based on the opacity treatment recommendation to treat the opacity.

9. A method for using in treatment of an opacity within an eye, the method comprising:
receiving input data, the input data comprising image data of the eye being indicative of a convergence spot of first and second aiming beams entering the eye and converging at a focal plane location of an opacity within the eye wherein, the first and second aiming beams cross each other to form first and second spots on the retina by respectively the first and second aiming beams at a distance spaced from one another;
processing the input data, the processing comprises:
processing the image data and determining an inter-spot distance between the first and second spots formed on the retina; and
utilizing the inter-spot distance and a model of the eye and determining a first distance between the opacity and the retina and a second distance between the opacity and the crystalline lens of the eye; and
generating output data indicative of opacity treatment recommendation based on the first and second distances.

10. The method according to claim 9, wherein said opacity treatment recommendation is to treat the opacity when both of said first and second distances are above a predetermined value, and to refrain from treating the opacity when either of the first or second distances is below the predetermined value.

11. The method according to claim 10 wherein said predetermined value is up to 3 millimeters.

12. The method according to claim 9, wherein said receiving of the input data comprises receiving data indicative of a contact lens used in front of the eye when said image data of the eye is acquired, said processing of the input data comprises processing the data indicative of the contact lens for determining the first and second distances.

13. The method according to claim 12, comprising providing data of a plurality of contact lenses, enabling a user to choose the contact lens to use.

14. The method according to claim 9, wherein said receiving of the input data comprises receiving eye prescription data of an individual undergoing the treatment, said processing of the input data comprises processing the eye prescription data for determining the first and second distances.

15. A method of claim 9, the method further comprising:
generating and directing first and second aiming beams such as to converge at location of an opacity within the eye and form respective first and second spots on the retina of the eye,
acquiring the input data of image data of the eye, the image data comprising data indicative of trajectories of the first and second aiming beams within the eye, and
irradiating the opacity with a laser treatment beam in order to destroy and remove the opacity upon determining from the output data indicative of opacity treatment recommendation that both of said first and second distances are above a predetermined value.

16. The method according to claim 15, wherein said irradiating of the opacity is done automatically upon determining that both of said first and second distances are above the predetermined value.
